(19) United States
(12) Patent Application Publication
(10) Pub. No.: US 2026/0058002 A1
(43) Pub. Date: Feb. 26, 2026

(54) PROVISION OF A GRAPHICAL DISPLAY OF MEDICAL IMAGE DATA OF AN EXAMINATION OBJECT

(71) Applicant: Siemens Healthineers AG, Forchheim (DE)

(72) Inventor: Alois Regensburger, Poxdorf (DE)

(21) Appl. No.: 19/308,175

(22) Filed: Aug. 22, 2025

(30) Foreign Application Priority Data
Aug. 23, 2024 (DE) 10 2024 208 054.3

(51) Int. Cl. G16H 30/20; G16H 30/40

(52) U.S. Cl. CPC G16H 30/20; G16H 30/40

(57) ABSTRACT

A method for provision of a graphical display of medical image data of an examination object includes acquiring a perception parameter of an observer. The perception parameter characterizes a visual limitation of the observer. The method includes providing the medical image data using a medical imaging device, and providing the graphical display of the medical image data using a display unit. The providing of the medical image data and/or the providing of the graphical display of the medical image data is adapted as a function of the perception parameter.

### This application claims the benefit of German Patent Application No. DE 10 2024 208 054.3, filed on Aug. 23, 2024, which is hereby incorporated by reference in its entirety.

### BACKGROUND

The present embodiments relate to provision of a graphical display of medical image data of an examination object.

In interventional medicine, image data (e.g., x-ray images) of rapidly varying (e.g., moving) anatomical structures, such as, for example, the heart and/or the lungs of an examination object are frequently generated and are shown in a sequence. This image data makes it possible for those carrying out the procedure to follow and/or to control a location and movement of medical objects within the examination object (e.g., a catheter and/or a stent). The publication by Von Haarlem CS, et al., The speed of sight: Individual variation in critical flicker fusion thresholds, (2024), PLOS ONE 19(4): e0298007, discloses that an astonishingly high variability exists between different people for the temporal visual processing capability of image signals. While image frame rates higher than 35 Hz are already no longer perceived for a few observers, this perception limit lies at around 60 Hz for other observers. In order to guarantee a fluid and clear perception of the sequence, the image data is frequently recorded with a high recording rate (e.g., 30 Hz or 60 Hz). The disadvantage of this, however, is that both the examination object and also the person carrying out the procedure are exposed to a high load, especially a high radiation dose, which may entail health risks. In order to reduce the load, especially the high radiation dose, standard protocols with predefined recording rates may be used, for example, where the recording rates have to be adapted manually to the respective person carrying out the procedure (e.g., to the respective observer).

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an improved provision of a graphical display of image data tailored to the observer is provided.

Independent of the grammatical term usage of a specific person-related term, individuals with male, female, or other gender identities should be included within the term.

In a first aspect, the present embodiments relate to a method for provision of a graphical display of medical image data of an examination object. In a first act, a perception parameter of an observer is acquired. In this case, the perception parameter characterizes a visual limitation of the observer. In a further act, the medical image data is provided by a medical imaging device. In a further act, the graphical display of the medical image data is provided by a display unit. In this case, the provision of the medical image data and/or the provision of the graphical display of the medical image data is adapted as a function of the perception parameter.

The acquisition of the perception parameter may, for example, include an acquisition and/or reading out of a computer-readable data memory and/or a receipt from a data storage unit (e.g., a database). As an alternative or in addition, the perception parameter may be acquired with the aid of a user input (e.g., a user input of the observer) and/or based on a physiological parameter of the observer.

The observer may, for example, be a female or male doctor and/or a medical operator who is to look at or is looking at the graphical display of the image data.

The perception parameter may characterize a (e.g., individual and/or person-specific) visual limitation (e.g., a restriction of a visual perception, such as a processing capacity) of the observer. The visual perception of the observer may be characterized by temporal and/or spatial and/or light-specific features. The temporal features of the perception may, for example, include a perception rate and/or temporal processing capacity and/or a reaction time of the observer. The spatial features of the perception may, for example, include a spatial resolution (e.g., distance-dependent in relation to the observer) and/or a spatial perception range and/or a focus range and/or a spatial processing capacity of the observer. The light-specific features of the perception of the observer may, for example, include a light wavelength and/or a light wavelength range and/or a color perception and/or contrast perception.

The perception parameter may characterize one or more features of the visual limitation of the observer.

The provision of the medical image data may, for example, include an acquisition and/or reading out from a computer-readable data memory and/or a receipt from a data storage unit (e.g., a database). In this case, the medical image data may be recorded by the medical imaging device. The medical image data may further be provided by a processing unit of a medical imaging device for recording the image dataset. The medical imaging device may, for example, include a Magnetic Resonance Tomography (MRT) system and/or a Computed Tomography (CT) system and/or a medical x-ray device (e.g., a medical C-arm x-ray device), and/or an ultrasound device and/or a Positron Emission Tomography (PET) system. The medical x-ray device may, for example, be embodied as an interventional x-ray device.

The medical image data may include a representation (e.g., a depiction and/or a model) of the examination object. In this case, the medical image data of the examination object may be spatially resolved in a two-dimensional (2D) and/or three-dimensional (3D) manner. Further, the medical image data may be temporally resolved (e.g., depict a scene). The image data may have a number of image points (e.g., pixels and/or voxels), each with at least one image value (e.g., a number of image values, such as time intensity curves). In this case, the image values of the image points may each represent (e.g., depict and/or model) a subvolume of the examination object.

The provision (e.g., a recording and/or processing) of the medical image data may be adapted as a function of the perception parameter. For example, at least one (e.g., temporal and/or spatial and/or light-specific) feature of the medical image data that relates to the visual limitation (e.g., the visual perception) of the observer may be identified with the aid of the perception parameter. The adaptation of the provision of the image data may include an adaptation of at least one provision parameter (e.g., of a recording parameter and/or processing parameter) of the medical image data. In this case, the at least one provision parameter may be adapted such that the at least one feature of the medical image data lies within the visual limitation (e.g., within the visual perception) of the observer. In one embodiment, the medical image data is provided (e.g., recorded and/or processed) by the at least one adapted provision parameter.

In accordance with a first variant, the graphical display of the medical image data may include a 2D representation of the medical image data (e.g., a projection of the medical image data onto a virtual display surface). In this case, the provision of the graphical display of the medical image data may include a display and/or projection of the graphical display onto a display surface of the display unit. The display unit may, for example, include a screen and/or monitor and/or projector and/or smart glasses.

In accordance with a second variant, the graphical display of the medical image data may include a 3D representation of the medical image data (e.g., a stereoscopic representation). In this case, the provision of the graphical display of the medical image data may include displaying and/or projecting the graphical display, in an at least part overlay and/or nesting, for example, on a display surface of the display unit or on separate display surfaces of the display unit. The display unit may, for example, include a screen and/or monitor and/or projector and/or smart glasses.

The provision of the graphical display of the medical image data may be adapted as function of the perception parameter. For example, at least one (e.g., temporal and/or spatial and/or light-specific) feature of the graphical display of the medical image data that relates to the visual limitation (e.g., the visual perception) of the observer may be identified with the aid of the perception parameter. The adaptation of the provision of the graphical display of the medical image data may include an adaptation of at least one display parameter of the graphical display and/or of the display unit. In this case, the at least one display parameter may be adapted such that the at least one feature of the graphical display of the medical image data lies within the visual limitation (e.g., within the visual perception) of the observer.

In one embodiment, the graphical display of the medical image data is provided (e.g., displayed) in accordance with the at least one adapted display parameter. The display parameter may, for example, include a frame rate and/or color depth and/or color map and/or a color spectrum and/or a contrast of the graphical display.

The method of the present embodiments may make possible an improved provision of a graphical display of the medical image data that is adapted to the observer.

In a further form of embodiment of the method, the provision of the medical image data may include a recording of the image data by the imaging device and/or a processing of the medical image data by a processing unit.

The recording of the medical image data may include an acquisition of the medical image data by the imaging device. For example, the imaging device may include a source (e.g., an x-ray source) and a detector (e.g., an x-ray detector). In this case, the recording of the medical image data may include emission of x-ray radiation by the source for fluoroscopy of the examination object arranged between the source and the detector. The x-rays may further be detected (e.g., acquired) by the detector after an interaction with the examination object. The medical image data may be acquired (e.g., reconstructed) with the aid of the detected x-rays. The source and the detector may, for example, be arranged in a defined arrangement in relation to one another (e.g., on a common C-arm). In this case, the defined arrangement of source and detector may be arranged in a movable (e.g., translatable and/or rotatable) manner in relation to the examination object.

As an alternative or in addition, the provision of the medical image data may include a processing of the image data (e.g., a reconstruction and/or filtering of the image data and/or an application of a correction function, such as a function for correction of artifacts, and/or an application of a logical and/or arithmetic operation to the medical image data) by a processing unit. The correction function may be embodied, for example, for correction of metal and/or movement artifacts. In this case, the recorded and/or processed medical image data may be provided to the display unit by the processing unit.

The form of embodiment, by the adaptation of the recording and/or processing of the medical image data to the perception parameter, may make possible an optimized provision of the graphical display that is tailored to the visual needs of the observer.

In a further form of embodiment of the method, the recording and/or processing of the medical image data may be adapted as a function of the perception parameter.

In one embodiment, the recording of the medical image data may be adapted as a function of the perception parameter. At least one (e.g., temporal and/or spatial and/or light-specific) feature of the medical image data that relates to the visual limitation (e.g., the visual perception) of the observer may be identified, for example, with the aid of the perception parameter. The adaptation of the recording of the medical image data may include an adaptation of at least one recording parameter of the imaging device. The at least one recording parameter may, for example, include a recording rate and/or recording duration and/or an imaging area and/or a spatial resolution and/or a dynamic of the image data and/or a gray value resolution of the image data and/or a predefined Signal-to-Noise Ratio (SNR) and/or a dose (e.g., an x-ray dose) per recording. In this case, the at least one recording parameter may be adapted such that the at least one feature of the medical image data lies within the visual limitation (e.g., within the visual perception) of the observer. In one embodiment, the medical image data is recorded by the at least one adapted recording parameter.

As an alternative or in addition, the processing of the medical image data may be adapted as a function of the perception parameter. The adaptation of the processing of the medical image data may include an adaptation of at least one processing parameter of the processing unit. The at least one processing parameter may, for example, predetermine a spatial resolution (e.g., a binning) and/or an interpolation and/or coloration and/or color removal and/or contrasting and/or (e.g., spatial and/or temporal) filtering and/or an edge setting and/or a temporal intermediate image interpolation and/or a denoising for the processing of the medical image data. The at least one processing parameter may further predetermine a brightness and/or Gamma curve and/or transfer curve of the graphical display. In this case, the at least one processing parameter may be adapted such that the at least one feature of the medical image data and/or of the graphical display of the medical image data lies within the visual limitation (e.g., within the visual perception) of the observer. In one embodiment, the medical image data is processed by the at least one adapted processing parameter.

The adaptation of the recording and/or processing of the medical image data as a function of the perception parameter enables the graphical display to be adapted optimally to the visual needs of the observer. For example, the recording and/or processing of the medical image data as a function of the perception parameter (e.g., as a boundary condition) may be optimized. This may lead to an improved detectability and appraisal of the medical image data (e.g., of anatomical structures and/or dynamic sequences and/or pathological changes).

In a further form of embodiment of the method, the adaptation of the recording of the medical image data may include an adaptation of a recording rate as a function of the perception parameter.

The adaptation of the recording rate may include an increase or decrease in the recording rate. The recording rate may identify a number of images to be recorded per unit of time (e.g., images per second). For example, the recording rate may be increased in order to image spatial and/or temporal changes of the examination object that are faster than a visual perception rate of the observer. The graphical display of the medical image data may subsequently have a slowed-down representation of the graphical display of the medical image data (e.g., a slow motion). This may, for example, be useful when investigating fast movements of cells, organs, or tissues, which would otherwise not be visible to the human eye. The slowed-down representation enables the observer to analyze the movement more precisely and to recognize possible anomalies. In one embodiment, the image quality and the temporal resolution of the medical image data may be increased by this, without a visual limitation of the observer being exceeded by this.

As an alternative or in addition, the recording rate may be adapted to correspond to the visual perception rate of the observer (e.g., decreased or increased). For example, the recording rate may be compared with the perception rate and adapted to the perception rate. If the perception rate of the observer is below the recording rate, the recording rate may be decreased, for example. This may be advantageous, for example, when no or only a comparatively slow temporal and/or spatial change of the examination object in the image data to be recorded is to be depicted. This may enable a load on the examination object (e.g., an x-ray dose) to be minimized.

The adaptation of the recording rate as a function of the perception parameter may change the temporal resolution of the medical image data. Further, the graphical display of the medical image data may adapt to the visual perception rate of the observer. This may lead to an improved detectability and appraisal of spatial and/or temporal changes of the examination object.

In a further form of embodiment of the method, the adaptation of the processing of the medical image data may include at least one of the following acts as a function of the perception parameter: a slowing-down of a sequence within the medical image data; an interpolation of a sequence within the medical image data of a sequence within the medical image data; and/or a highlighting of anatomical and/or geometrical features in the medical image data as a function of the perception parameter.

The sequence within the medical image data may include a plurality of consecutive images that depict a temporal change of the examination object. The processing of the medical image data may, for example, include a slowing-down (e.g., temporal expansion) and/or interpolation of a sequence within the medical image data when the perception parameter predetermines a lower perception rate of the observer compared to a recording rate of the medical image data and/or a rate of change of an imaged change in the examination object. This may improve the ability to perceive the sequence for a slow-sighted observer. The sequence may further be displayed in the graphical display of medical image data more fluidly and more continuously. The processing of the medical image data may further include a highlighting of anatomical and/or geometrical features in the medical image data (e.g., when the perception parameter predetermines a restricted color visibility and/or contrast visibility and/or visual acuity of the observer). The highlighting of the anatomical and/or geometrical features of the medical image data may, for example, include an intensification of an image contrast of the medical image data. This enables the geometrical and/or anatomical features within the visual limitation of the observer to be shown graphically more plainly and/or clearly, for example. In this way, an ability to distinguish visually between the anatomical and/or geometrical features within the graphical display may be improved.

In a further form of embodiment of the method, a number of recording protocols with respective recording parameters may be provided for recording of the medical image data. In this case, at least one recording protocol from the number of recording protocols is identified based on the perception parameter. Further, the at least one recording protocol may make a recording of the medical image data possible taking into account the visual limitation of the observer. In this case, the medical image data may be recorded based on the at least one recording protocol.

The provision of the number of recording protocols may, for example, include an acquisition and/or reading out of a computer-readable data memory and/or a receipt from a data storage unit (e.g., a database). The number of recording protocols may further be acquired with the aid of a user input using an input unit.

The number of recording protocols provided may each be suitable for recording the medical image data (e.g., for fulfilling a predefined recording objective). The number of recording protocols may each include one or more (e.g., different) recording parameters for recording the medical image data. In this case, the values of the recording parameter of the number of recording protocols may differ at least in part.

The identification of the at least one recording protocol from the number of recording protocols based on the perception parameter may include an assessment of a suitability of medical image data able to be recorded using the respective recording protocol for a visual perceptibility in accordance with the perception parameter. In this case, for each of the number of recording protocols, a respective suitability value is identified based on the perception parameter. With the aid of a comparison of the suitability values of the number of recording protocols, the at least one recording protocol (e.g., a number of selected recording protocols of the number of recording protocols that have a comparatively highest, or maximal, suitable value compared to the other suitability values) may be identified. As an alternative, with the aid of a comparison of the suitability values of the number of recording protocols with a predetermined threshold value, the at least one recording protocol(e.g., a number of selected recording protocols of the number of recording protocols) that have a suitability value above the predetermined threshold value are identified. In this case, a comparatively higher suitability value may characterize a higher (e.g., better) suitability of the recording protocol for recording medical image data in relation to the perception parameter.

In one embodiment, the at least one identified recording protocol of the number of recording protocols may make possible a recording of medical image data taking into account the visual limitation of the observer (e.g., without exceeding the visual limitation of the observer). A workflow tip having an identification of the at least one identified recording protocol (e.g., an optical highlighting, such as an optical marking, and/or a text) may further be provided.

In one embodiment, the medical image data may be recorded based on the at least one recording protocol. For example, the medical image data may be recorded using the identified recording protocol or one of the identified recording protocols. In this case, the recording parameter of the medical imaging device may be set in accordance with the respective identified recording protocol for recording of the medical image data (e.g., manually, semi-automatically, or automatically).

The form of embodiment makes possible an efficient selection of at least one recording protocol from the number of recording protocols that is suitable for the observer and a recording objective (e.g., the imaging of a temporal change in the examination object).

In a further form of embodiment of the method, the perception parameter may characterize at least one of the following visual limitations of the observer: a perception rate, a color visibility, a contrast visibility, a visual acuity, and/or a reaction time.

The perception rate may characterize a number of images per unit of time that the observer may perceive visually. The color visibility may characterize a capability of the observer to distinguish between various colors (e.g., when arranged adjacent to one another). Contrast visibility may characterize the capability of the observer to recognize differences in brightness. Visual acuity of the observer may characterize the capability of the observer to distinguish between two neighboring image points of the graphical display of the medical image data (e.g., to resolve them spatially). Reaction time of the observer may characterize a time needed by the observer in order to react to a visual stimulus.

The various aspects of the perception parameter (e.g., the perception rate and/or color visibility and/or contrast visibility and/or visual acuity and/or reaction time) may influence one another by strengthening or weakening. In one embodiment, any mutual influencing of the various aspects of the perception parameter may be taken in consideration in the adaptation of the provision of medical image data and/or the provision of the graphical display of the medical image data.

The processing of the medical image data may make possible the adaptation of the graphical display of the medical image data to the visual limitation of the observer. The slowing-down and/or interpolation of a sequence within the medical image data enables the temporal change of the examination object to be displayed more fluidly and continuously. The highlighting of anatomical and/or geometrical features in the medical image data enables the ability to visually distinguish between the features within the graphical display to be improved.

In a further form of embodiment of the method, the medical image data may depict a temporal change of the examination object. In this case, a rate of change of the temporal change may be identified with the aid of the medical image data. Further, the provision of the medical image data and/or the provision of the graphical display of the medical image data may additionally be adapted as a function of the rate of change.

The temporal change may, for example, include a movement of at least one part (e.g., of an organ and/or of tissue) of the examination object and/or of a medical object and/or of a contrast medium in the examination object. The rate of change may characterize a speed of the temporal change depicted (e.g., of the movement). The identification of the rate of change may include an identification of a change of positioning (e.g., a speed of the change of positioning), of features (e.g., anatomical and/or geometrical features depicted in the medical image data). Anatomical features may, for example, include an anatomical landmark and/or a tissue boundary. Geometrical features may, for example, include a contour and/or marker structure.

In one embodiment, the provision (e.g., the recording and/or processing of the medical image data) may be adapted as a function of the perception parameter and the rate of change. In this case, the provision of the medical image data (e.g., of the provision parameters) may be adapted as a function of the perception parameter and the rate of change such that a recording rate of the temporal change is at least the same as or greater than the rate of change.

Further, the provision of the graphical display (e.g., of the display parameters) may be adapted as a function of the perception parameter and the rate of change. If the rate of change lies above a perception rate of the observer, the graphical display having a slowed down display of the sequence may be provided, for example, where the sequence depicts the temporal change of the examination object.

In one embodiment, the provision of the medical image data and/or of the graphical display of the medical image data may be adapted to the temporal change of the examination object in order to make possible a secure, fluid, and continuous visualization. The fact that the adaptation is based both on the rate of change of the temporal change and also on the perception parameter of the observer enables an individual and optimal representation to be provided.

In a further form of embodiment of the method, the medical image data may depict a temporal change of the examination object. In this case, a rate of change of the temporal change may be identified with the aid of the medical image data. Further, it may be identified with the aid of the rate of change and the perception parameter whether the temporal change depicted in the medical image data is able to be detected by the observer. Depending on the detectability of the temporal change depicted in the medical image data, a workflow tip may be provided.

The temporal change may, for example, include a movement of at least one part (e.g., of an organ and/or of tissue) of the examination object and/or of a medical object and/or of a contrast medium in the examination object. The rate of change may characterize a speed of the temporal change depicted (e.g., of the movement). The identification of the rate of change may include an identification of a change of positioning (e.g., a speed of the change of positioning) of features (e.g., anatomical and/or geometrical features depicted in the medical image data). Anatomical features may, for example, include an anatomical landmark and/or a tissue boundary. Geometrical features may, for example, include a contour and/or marker structure.

The identification of whether the temporal change depicted in the medical image data is able to be perceived by the observer may include a comparison of the rate of change with a perception rate and/or a reciprocal of the reaction time of the observer. If the result of the comparison is that the rate of change is greater than the perception rate and/or greater than the reciprocal of the reaction time of the observer, it may identify that the temporal change depicted is not able to be detected by the observer. Depending on the detectability of the temporal change depicted in the medical image data (e.g., at least on identification of the non-detectability), the workflow tip may be provided.

The workflow tip may, for example, include a graphical element (e.g., a text and/or a symbol) and/or an acoustic element (e.g., a tone and/or a melody and/or a speech output) and/or a haptic element (e.g., a vibration). The workflow tip may be provided by an output unit (e.g., the display unit and/or a loudspeaker and/or a haptic provision unit).

In one embodiment, the observer may be alerted by the workflow tip to a relevant temporal change of the examination object that the observer may otherwise overlook. Further, the workflow tip may be provided in various modalities (e.g., graphically and/or acoustically and/or haptically) in order to take account of requirements and preferences of the observer. The workflow tip may further feature a recommendation to the observer to look at the sequence once again in slow motion.

In a further form of embodiment of the method, the acquisition of the perception parameter may include an acquisition of a user input and/or an acquisition of a physiological parameter of the observer by an acquisition unit.

The acquisition unit may include an input unit for acquiring the user input. The input unit may, for example, include a keyboard and/or a pointer device and/or a touchscreen and/or an operating panel and/or a switch and/or a camera and/or a microphone. The input unit may be configured to detect the user input (e.g., the user input of the observer). The user input may include information about the perception parameter of the observer. The user input may, for example, include a speech input and/or a keyboard input and/or a touchscreen input and/or an input gesture and/or an eye movement (e.g., a focus point) of the observer. In one embodiment, the input unit may provide the perception parameter after the acquisition of the user input.

An advantage of this form of embodiment consists of the observer being able to provide the perception parameter that characterizes their visual perception of temporal changes using the user input itself.

The acquisition unit may further include a, for example, electromagnetic and/or optical and/or acoustic and/or mechanical sensor for acquisition of the physiological parameter of the observer. The sensor may be configured for (e.g., automatic) acquisition of the physiological parameter of the observer, The physiological parameter of the observer may characterize the visual limitation of the observer (e.g., indirectly or directly). The physiological parameter of the observer may, for example, characterize a property of one or of both eyes of the observer.

The proposed form of embodiment may facilitate the adaptation of the graphical display of the medical image data to the individual needs and preferences of the observer and improve the diagnostic accuracy and efficiency.

In a further form of embodiment of the method, the acquisition of the perception parameter may include a provision of at least one graphical test display by a display unit or a further display unit. In this case, the provision of the graphical test display may be based on a display parameter. In this case, the user input and/or the physiological parameter of the observer may be acquired during or after the provision of the graphical test display.

The graphical test display may be provided (e.g., displayed) by the display unit for provision of the graphical display or by a further display unit (e.g., based on a display parameter). In this case, the provision of the graphical test display may include a display and/or projection of the graphical test display on a display surface of the display unit or a display surface of the further display unit. The further display unit may, for example, include a screen and/or monitor and/or projector and/or smart glasses. The display parameter may predetermine properties of the graphical test display (e.g., a color space and/or a brightness and/or a contrast and/or a spatial resolution and/or a temporal resolution and/or a scaling and/or a display duration). The graphical test display may, for example, include a number (e.g., a sequence of digits) and/or a text (e.g., a sequence of letters) and/or a symbol and/or a pattern and/or an animation and/or medical test image data (e.g., including a scene).

In one embodiment, the user input and/or the physiological parameter may be acquired at a point in time during or after the provision (e.g., the display) of the graphical test display. For example, the user input and/or the physiological parameter may be acquired repeatedly and/or continuously during or after the provision of the graphical test display.

In one embodiment, the observer may be requested to provide the user input as a function of the graphical test display (e.g., at that moment, such as of a content of the graphical test display) by the acquisition unit (e.g., the input unit). The user input may feature information about a content of the graphical test display observed by the observer.

As an alternative or in addition, a physiological parameter of the observer may be acquired during or after the provision of the graphical test display (e.g., automatically) by the acquisition unit (e.g., the sensor). In one embodiment, the physiological parameter may characterize a (e.g., physiological) reaction of the observer to the graphical test display.

For the individual observer, a maximum perceivable perception rate ERₘₐₓ may be established. This may be undertaken, for example, by a flicker test or via other graphical displays. As an alternative, the performance and/or speed of reaction of the observer for various image rates may be established in a computer game or intervention simulator scenario. In one embodiment, the image rates of the graphical test display may be randomized or systematically varied and the capabilities of the observer analyzed depending thereon. Effective variation strategies in this case enable learning and tiredness effects of the observer to be statistically suppressed.

In one embodiment, the perception parameter may be identified with the aid of the user input and/or of the physiological parameter. The physiological parameter in this case may characterize a (e.g., physiological) reaction of the observer to the graphical test display (e.g., a pulse change and/or rate of blinking and/or eye movement and/or muscle activity). In one embodiment, the perception parameter may be identified (e.g., determined) with the aid of the user input and/or of the physiological parameter and taking into consideration the graphical test display (e.g., the content and/or the display parameter of the graphical test display).

An advantage of this form of embodiment consists of a specific identification of the visual Limitation of the observer being able to be made possible. This may facilitate an individual adaptation of the graphical display of the image data to the visual limitation of the observer and advantageously improve the quality and efficiency during the acquisition of the graphical display of medical image data.

In a further form of embodiment of the method, the user input may be detected during or after the provision of the graphical test display. In this case, the user input may be compared with the content of the graphical test display.

The comparison may include checking on the perception of the content of the graphical test display by the observer. Through this, the comparison may make possible an identification of the perception parameter. For example, with the aid of the comparison, a limitation of a color visibility and/or contrast visibility and/or visual acuity of the observer may be identified. For example, when the result of the comparison is that the contents of the graphical test display has not been detected or has not been fully detected by the observer, a visual limitation of the observer may be identified as a function of the associated display parameter of the graphical test display. Subsequently, based on the identified visual limitation of the observer, the perception parameter may be identified (e.g., determined).

An advantage of this form of embodiment consists of the graphical test display, the user input, the comparison, and the perception parameter making possible a specific identification of the visual limitation of the observer. This may facilitate an individual adaptation of the graphical display of the image data to the visual limitation of the observer and improve the quality and efficiency in the perception of the graphical display of the medical image data.

In a second aspect, the present embodiments relate to a system including a processing unit and a display unit. In this aspect, the processing unit is configured to acquire a perception parameter of an observer. The perception parameter characterizes a visual limitation of the observer. The processing unit is further configured to acquire medical image data of an examination object acquired by a medical imaging device. The display unit is configured to provide a graphical display of the medical image data of the examination object. The provision of the medical image data and/or the provision of the graphical display of the medical image data is adapted as a function of the perception parameter.

The system and its respective components may, for example, be configured to carry out a method for provision of a graphical display of medical image data of an examination object or of one of its forms of embodiment.

The advantages of the proposed system essentially correspond to the advantages of the proposed method. Features, advantages, or alternative forms of embodiment mentioned here may likewise be transferred to other claimed subject matter and vice versa.

In a further form of embodiment of the system, the system may also include a medical imaging device. In this case, the imaging device may be configured for recording the medical image data of the examination object.

The medical imaging device may, for example, include a Magnetic Resonance Tomography (MRT) system and/or a Computed Tomography (CT) system and/or a medical x-ray device (e.g., a medical C-arm x-ray device) and/or an ultrasound device and/or a Positron Emission Tomography (PET) system. The imaging device may further be configured to provide the medical image data to the processing unit.

In a third aspect, the present embodiments relate to a computer program product with a computer program that is able to be loaded directly into a memory of a processing unit, with program sections for carrying out all steps of a proposed method for provision of a graphical display of medical image data of an examination object when the program sections are executed by the processing unit.

The computer program product may, for example include software with a source code that still has to be compiled and linked or just has to be interpreted, or an executable software code, which is still to be loaded into the processing unit for execution. The computer program product enables the method for provision of a graphical display of medical image data of an examination object to be executed in a quick, identically repeatable and robust manner by a processing unit. The computer program product is configured so that it may carry out the method steps of the present embodiments by the processing unit.

The computer program product is stored on a computer-readable memory medium (e.g., a non-transitory computer-readable storage medium), for example, or is held on a network or server, from where the computer program product may be loaded into the processor of a processing unit that is directly connected to the processing unit or may be configured as a part of the processing unit. Further, control information of the computer program product may be stored on an electronically-readable data medium. The control information of the electronically-readable data medium may be configured such that, when the data medium is used in a processing unit, the control information carries out a method of the present embodiments. Examples of electronically-readable data media are a DVD, a magnetic tape, or a USB stick, on which electronically-readable control information (e.g., software) is stored. When this control information is read from the data medium and is stored in a processing unit, all forms of embodiment of the method described above may be carried out.

A largely software-based realization has the advantage that even processing units already used previously may be upgraded in a simple way by a software update in order to work according to the present embodiments. Such a computer program product, as well as the computer program, may, where necessary, include additional elements such as, for example, documentation and/or additional components, as well as hardware components, such as, for example, hardware keys (e.g., dongles, etc.) for use of the software.

### BRIEF DESCRIPTION OF THE DRAWINGS

Example embodiments are shown in the drawings and will be described in greater detail below. The same reference characters are used in different figures for the same features. In the figures:

FIGS. 1 to 6 show schematic diagrams of forms of embodiment of a method for provision of a graphical display of medical image data; and

FIGS. 7 and 8 show schematic diagrams of forms of embodiment of a system.

### DETAILED DESCRIPTION

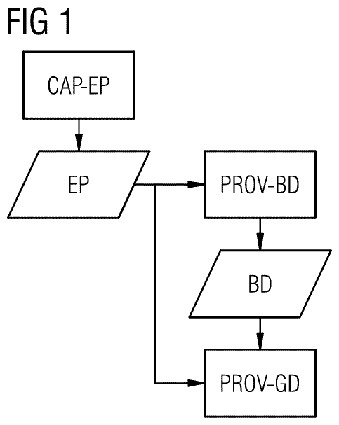

FIG. 1 shows a schematic diagram of a form of embodiment of a method for provision of a graphical display PROV-GD of medical image data BD of an examination object. In this case, a perception parameter EP of an observer may be acquired CAP-EP. The perception parameter EP may characterize a visual limitation of the observer. For example, the perception parameter EP may characterize a perception rate and/or a color visibility and/or a contrast visibility and/or a visual acuity and/or a reaction time of the observer. The medical image data BD may further be provided PROV-BD by a medical imaging device. Further, the graphical display of the medical image data BD may be provided PROV-GD by a display unit. In this case, the provision PROV-BD of the medical image data and/or the provision PROV-GD of the graphical display of the medical image data BD may be adapted as a function of the perception parameter EP.

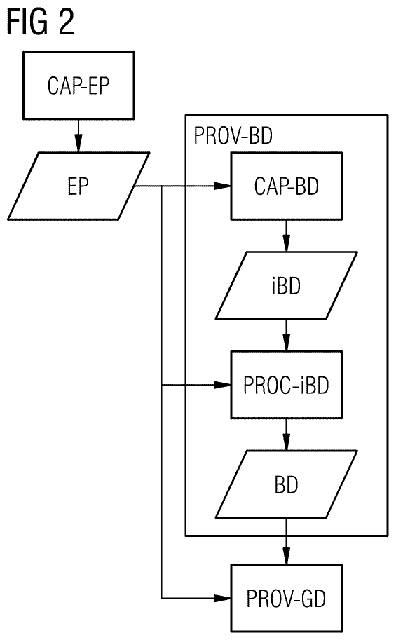

FIG. 2 shows a schematic diagram of a further form of embodiment of a method for provision PROV-GD of a graphical display of medical image data BD of an examination object. In this case, the provision of the medical image data PROV-BD may include a recording of the medical image data CAP-BD by the imaging device and/or a processing of the medical image data PROC-BD by a processing unit. For example, the recording of the medical image data CAP-BD and/or the processing of the medical image data PROC-BD may be adapted as a function of the perception parameter EP. For example, the adaptation of the recording of the medical image data CAP-BD may include an adaptation of a recording rate as a function of the perception parameter EP. For example, a maximum perceivable perception rate ERₘₐₓ may be established for the observer. In one embodiment, the perception parameter EP may feature information about the maximum perception rate ERₘₐₓ. The recording rate may be adapted as a function of the perception parameter EP such that (e.g., the maximum perception rate ERₘₐₓ is adapted) no recording with a higher recording than ERₘₐₓ takes place. The recording rate may, where necessary, be increased or decreased by a tolerance factor in relation to ERₘₐₓ. If a recording protocol, for example, has a recording rate of 60 Hz, and ERₘₐₓ lies below 60 Hz, the recording rate for the recording of the medical image data BD may be reduced to 45 Hz, for example.

Further, the adaptation of the processing of the medical image data PROV-BD may include a slowing down and/or interpolation of a sequence within the medical image data BD and/or a highlighting of anatomical and/or geometrical features in the medical image data BD as a function of the perception parameter EP.

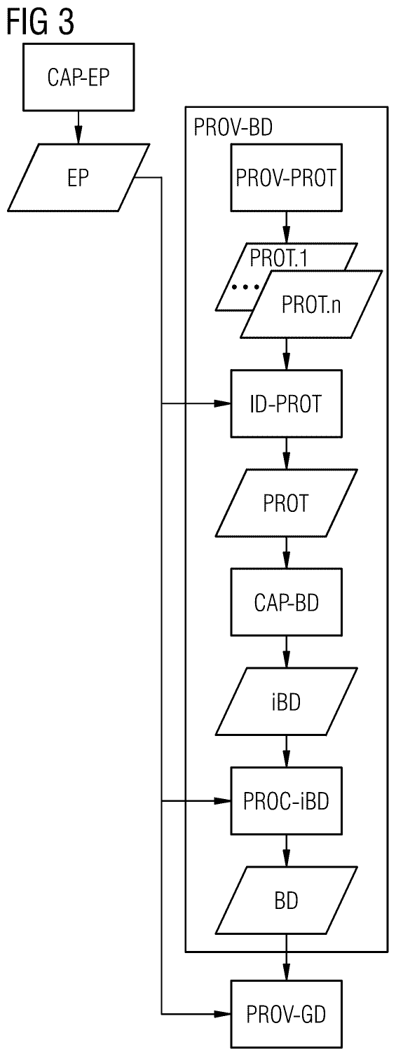

FIG. 3 shows a schematic diagram of a further form of embodiment of a method for provision of a graphical display PROV-GD of medical image data BD of an examination object. In this case, a number of (e.g., several; a number n) recording protocols PROT.1 to PROT.n, with respective recording parameters for recording the medical image data CAP-BD may be provided PROV-PROT. In one embodiment, at least one recording protocol PROT from the number of recording protocols PROT.1 to PROT. n may be identified ID-PROT based on the perception parameter EP. In this case, the at least one recording protocol PROT may make possible a recording of the medical image data CAP-BD taking into account the visual limitation of the observer. The medical image data CAP-BD may further be recorded based on the at least one recording protocol PROT.

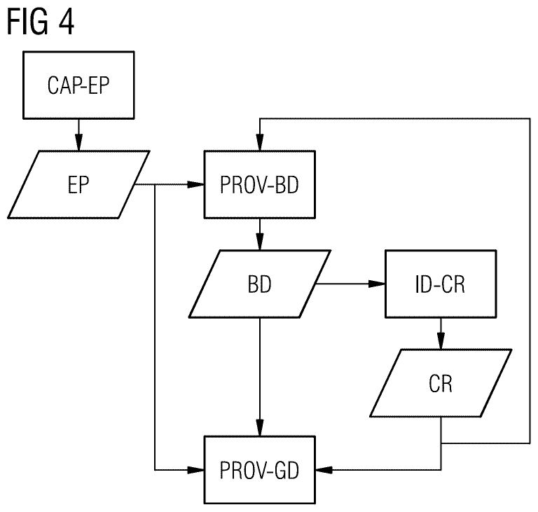

FIG. 4 shows a schematic diagram of a further form of embodiment of a method for provision PROV-GD of a graphical display of medical image data BD of an examination object. In this case, the medical image data BD may depict a temporal change of the examination object. A rate of change CR of the temporal change may further be identified ID-CR with the aid of the medical image data BD. In this case, the provision PROV-BD of the medical image data and/or the provision PROV-GD of the graphical display of the medical image data BD may be adapted additionally as a function of the rate of change CR. It may further be identified with the aid of the rate of change CR and the perception parameter EP whether the temporal change depicted in the medical image data BD is able to be detected by the observer. A workflow tip may be provided as a function of the detectability of the temporal change depicted in the medical image data (BD).

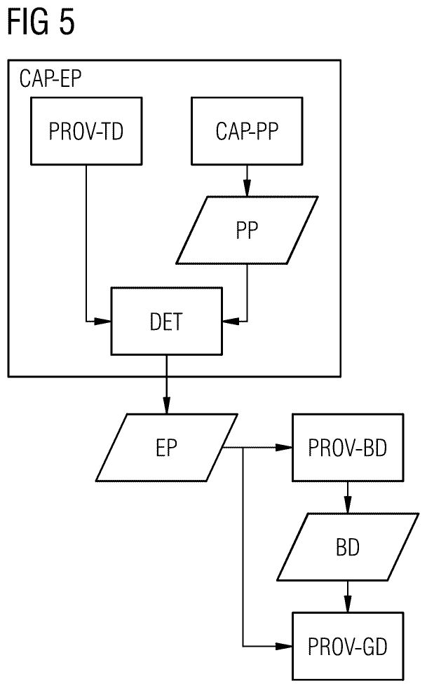

FIG. 5 shows a schematic diagram of a further form of embodiment of a method for provision PROV-GD of a graphical display of medical image data BD of an examination object. In this case, the acquisition CAP-EP of the perception parameter EP may include a provision PROV-TD of at least one graphical test display by the display unit or a further display unit. The provision PROV-TD of the graphical test display may further be based on a display parameter. In one embodiment, a physiological parameter PP of the observer may further be acquired CAP-PP by an acquisition unit (e.g., a sensor) during or after the provision PROV-TD of the graphical test display. In this case, the perception parameter EP may be determined DET based on the physiological parameter PP.

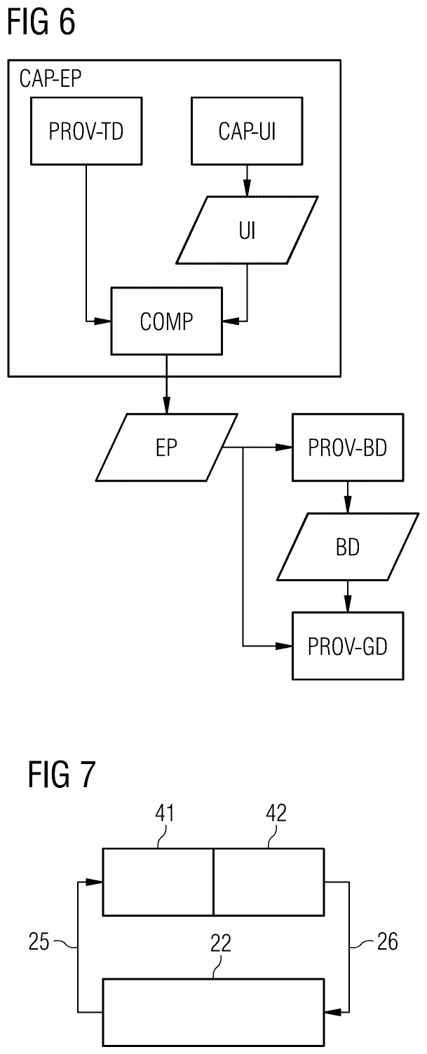

FIG. 6 shows a schematic diagram of a further form of embodiment of a method for provision PROV-GD of a graphical display of medical image data BD of an examination object. In this case, the acquisition CAP-EP of the perception parameter EP may include a provision PROV-TD of at least one graphical test display by the display unit or a further display unit. The provision PROV-TD of the graphical test display may further be based on a display parameter. In one embodiment, a user input UI may further be acquired CAP-UI by the acquisition unit (e.g., an input unit) during or after the provision PROV-TD of the graphical test display. In this case, the user input UI may be compared COMP with the contents of the graphical test display. In one embodiment, the perception parameter EP may be identified as a function of the comparison.

FIG. 7 shows a schematic diagram of a form of embodiment of a system. The system may include a processing unit 22 and a display unit 41 (e.g., a monitor and/or a display and/or a projector). The system may further include an acquisition unit 42 (e.g., a sensor and/or an input unit). The input unit may, for example, include a keyboard. The sensor may be configured, for example, as an electromagnetic and/or optical and/or acoustic and/or mechanical sensor. The acquisition unit 42 may be integrated into the display unit 41. For example, the input unit may be integrated, with a capacitive and/or resistive input display, into the display unit 41. The sensor may, for example, be arranged on the observer or spaced apart from the observer (e.g., on the display unit 41). The processing unit 22 may be configured to detect CAP-EP the perception parameter EP of the observer (e.g., using an interface). The processing unit 22 may further be configured to detect CAP-BD medical image data BD of an examination object, recorded by a medical imaging device. The display unit 41 may further be configured to provide PROV-GD a graphical display of the medical image data BD of the examination object. For this, the processing unit 22 may send a signal 25 to the display unit 41.

The acquisition unit 42 (e.g., the input unit) may be configured for detection of a user input UI. The acquisition unit 42 (e.g., the sensor) may further be configured for detection of the physiological parameter PP of the observer. For this, the acquisition unit 42 may, for example, send a signal 26 to the processing unit 22 (e.g., the interface of the processing unit 22). The processing unit 22 may be configured to acquire CAP-EP the perception parameter EP of the observer with the aid of the user input UI and/or of the physiological parameter PP.

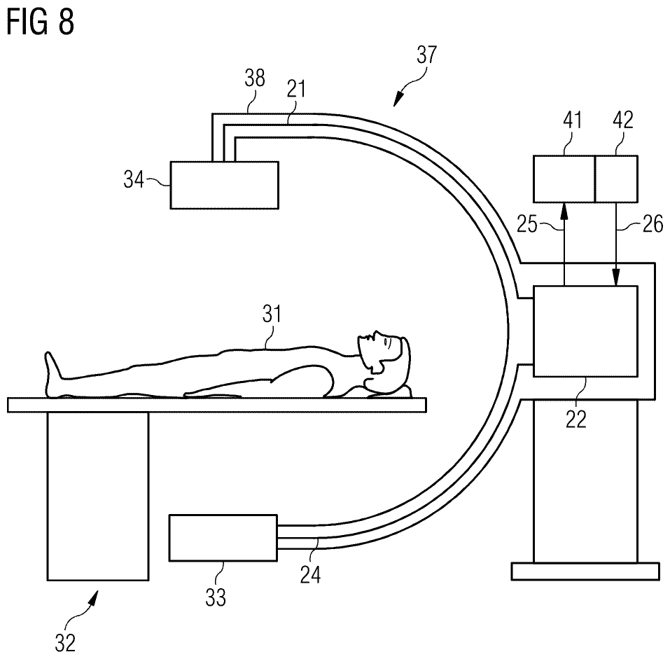

FIG. 8 shows a schematic diagram of a further form of embodiment of a proposed system. The system in this case may further include a medical imaging device 37 (e.g., a C-arm x-ray device). The medical imaging device 37 may have a source 33 (e.g., an x-ray source) and a detector 34 (e.g., an x-ray detector) that are arranged in a defined arrangement on a C-Arm 38. The C-arm 38 may be arranged movably about one or more axes.

For acquisition CAP-BD of the medical image data of the examination object 31 positioned on a patient support apparatus 32, the processing unit 22 may send a signal 24 to the source 33. In response, the source 33 may emit a ray bundle (e.g., an x-ray bundle). When the x-ray bundle, after an interaction with the examination object 31, strikes an x-ray-sensitive surface of the detector 34, the detector 34 may send a signal 21 to the processing unit 22 (e.g., provide PROV-BD the medical image data BD to the processing unit 22). The processing unit 22 may detect (e.g., receive) the medical image data of the examination object 31 with the aid of the signal 21.

The schematic representations contained in the figures described do not depict any sort of scale or size relationships.

It is pointed out in conclusion that the method described in detail above, as well as the apparatuses shown, merely involve example embodiments that may be modified by the person skilled in the art in a very wide variety of ways without departing from the field of the invention. Further, the use of the indefinite article “a” or “an” does not exclude the features concerned also being able to be present a number of times. Likewise, the terms “unit” and “element” do not exclude the components concerned consisting of a number of interacting part components, which, where necessary, may also be spatially distributed.

The expression “based on” may be understood in the context of the present application, for example, in the sense of the expression “using. ” For example, a formulation, according to which a first feature is created (e.g., alternatively, established, determined, etc.) based on a second feature, does not exclude that the first feature may be created (e.g., alternatively, established, determined, etc.) based on a third feature.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for provision of a graphical display of medical image data of an examination object, the method comprising:
acquiring a perception parameter of an observer, wherein the perception parameter characterizes a visual limitation of the observer;
providing the medical image data using a medical imaging device; and
providing the graphical display of the medical image data using a display unit,
wherein providing the medical image data, providing the graphical display of the medical image data, or providing the medical image data and providing the graphical display of the medical image data are adapted as a function of the perception parameter.

2. The method of claim 1, wherein providing the medical image data comprises acquiring the medical image data using the medical imaging device, processing the medical image data using a processing unit, or a combination thereof.

3. The method of claim 2, wherein acquiring the medical image data, processing the medical image data, or acquiring the medical image data and processing the medical image data are adapted as a function of the perception parameter.

4. The method of claim 3, wherein the adaptation of the acquiring of the medical image data comprises an adaptation of a recording rate as a function of the perception parameter.

5. The method of claim 3, wherein the adaptation of the processing of the medical image data comprises:
a slowing-down of a sequence within the medical image data as a function of the perception parameter;
an interpolation of a sequence within the medical image data as a function of the perception parameter;
a highlighting of anatomical, geometrical, or anatomical and geometrical features in the medical image data as a function of the perception parameter; or
any combination thereof.

6. The method of claim 2, further comprising:
providing a number of recording protocols with respective recording parameters for acquiring the medical image data; and
identifying at least one recording protocol from the number of recording protocols based on the perception parameter,
wherein the at least one recording protocol makes possible an acquisition of the medical image data taking into account the visual limitation of the observer, and
wherein the acquisition of the medical image data is undertaken based on the at least one recording protocol.

7. The method of claim 1, wherein the perception parameter characterizes a visual limitation of the observer, the visual limitation of the observer being a perception rate, a color visibility, a contrast visibility, a visual acuity, a reaction time, or any combination thereof.

8. The method of claim 1, wherein the medical image data depicts a temporal change of the examination object,
wherein a rate of change of the temporal change is identified with the aid of the medical image data, and
wherein the providing of the medical image data, the providing of the graphical display of the medical image data, or the providing of the medical image data and the providing of the graphical display of the medical image data are also adapted as a function of the rate of change.

9. The method of claim 1, wherein the medical image data depicts a temporal change of the examination object,
wherein a rate of change of the temporal change is identified with the aid of the medical image data, and
wherein the method further comprises:
identifying, with the aid of the rate of change and the perception parameter, whether the temporal change depicted in the medical image data is perceivable by the observer; and
providing a workflow tip as a function of an ability to perceive the temporal change depicted in the medical image data.

10. The method of claim 1, wherein acquiring the perception parameter comprises acquiring a user input, acquiring a physiological parameter of the observer using an acquisition unit, or a combination thereof.

11. The method of claim 10, wherein acquiring the perception parameter comprises providing at least one graphical test display using the display unit or a further display unit,
wherein providing the at least one graphical test display is based on a display parameter, a provision parameter, or the display parameter and the provision parameter, and
wherein the user input, the physiological parameter, or the user input and the physiological parameter of the observer are acquired during or after the providing of the graphical test display.

12. The method of claim 11, wherein the user input is acquired during or after the providing of the graphical test display,
wherein the user input is compared with contents of the graphical test display, and
wherein the perception parameter is identified as a function of the comparison.

13. A system comprising:
a processing unit; and
a display unit,
wherein the processing unit is configured to:
acquire a perception parameter of an observer, wherein the perception parameter characterizes a visual limitation of the observer; and
acquire medical image data of an examination object recorded by a medical imaging device,
wherein the display unit is configured to provide a graphical display of the medical image data, and
wherein the acquisition of the medical image data, the provision of the graphical display of the medical image data, or the acquisition of the medical image data and the provision of the graphical display of the medical image data are adapted as a function of the perception parameter.

14. The system of claim 13, further comprising the medical imaging device,
wherein the imaging device is configured to record the medical image data of the examination object.

15. A non-transitory computer-readable storage medium that stores instructions executable by one or more processors for provision of a graphical display of medical image data of an examination object, the instructions comprising:
acquiring a perception parameter of an observer, wherein the perception parameter characterizes a visual limitation of the observer;
providing the medical image data using a medical imaging device; and
providing the graphical display of the medical image data using a display unit,
wherein providing the medical image data, providing the graphical display of the medical image data, or providing the medical image data and providing the graphical display of the medical image data are adapted as a function of the perception parameter.

16. The non-transitory computer-readable storage medium of claim 15, wherein providing the medical image data comprises acquiring the medical image data using the medical imaging device, processing the medical image data using a processing unit, or a combination thereof.

17. The non-transitory computer-readable storage medium of claim 16, wherein acquiring the medical image data, processing the medical image data, or acquiring the medical image data and processing the medical image data are adapted as a function of the perception parameter.

18. The non-transitory computer-readable storage medium of claim 17, wherein the adaptation of the acquiring of the medical image data comprises an adaptation of a recording rate as a function of the perception parameter.
